(19) United States
(12) Patent Application Publication
(10) Pub. No.: US 2026/0207089 A1
(43) Pub. Date: Jul. 23, 2026

(54) METHOD AND APPARATUS FOR NON-INVASIVELY MEASURING BLOOD CIRCULATORY HEMOGLOBIN

(71) Applicant: Becton, Dickinson and Company, Franklin Lakes, NJ (US)

(72) Inventors: Anusha Alathur Rangarajan, Irvine, CA (US); Brennan Michael Schneider, Irvine, CA (US); Robert J. Kopotic, Jamul, CA (US)

(21) Appl. No.: 19/139,775

(22) PCT Filed: Dec. 14, 2023
(86) PCT No.: PCT/US2023/083954

§ 371 Date: Jun. 16, 2025

Related U.S. Application Data
(60) Provisional application No. 63/387,926, filed on Dec. 16, 2022.

(51) Int. Cl. A61B 5/1455; A61B 5/0205; A61B 5/021; A61B 5/145

(52) U.S. Cl. CPC A61B 5/1455; A61B 5/0205; A61B 5/02108; A61B 5/14546; A61B 2560/0223

(57) ABSTRACT

A method and system of non-invasively determining continuous total hemoglobin data is provided. The method includes: a) using a near infra-red spectrophotometric (NIRS) sensing device on a continuous basis to sense a subject's tissue, wherein NIRS signals are produced from the sensing and are representative of total hemoglobin (THb); b) providing a body mass index (BMI) value of the subject; c) selectively calibrating the NIRS sensing device using a reference absolute THb value acquired from the subject, wherein the selective calibration includes calibrating or not calibrating the NIRS sensing device based on the BMI value; and d) determining continuous THb data using the produced NIRS signals.

### 1. TECHNICAL FIELD

This invention relates to methods and apparatus for determining blood circulatory hemoglobin values in general, and to non-invasive methods and apparatus for determining blood circulatory hemoglobin values that utilize performance criteria in particular.

### 2. BACKGROUND INFORMATION

The molecule that carries the oxygen in the blood is hemoglobin. Oxygenated hemoglobin (i.e., oxyhemoglobin or HbO₂) and deoxygenated hemoglobin (i.e., deoxyhemoglobin or Hb) are the predominate types of hemoglobin present in blood, but blood may contain other types of hemoglobin (e.g., carboxyhemoglobin (COHb), methemoglobin (MetHb)) which are typically in much smaller amounts. The term “total hemoglobin” used herein refers to the sum of HbO₂ and Hb, and is proportional to relative blood volume changes, provided that the hematocrit (i.e., gravimetric value) or hemoglobin concentration (i.e., volumetric) of the blood is unchanged.

Near-infrared spectroscopy (NIRS) is an optical spectrophotometric method of continually monitoring tissue parameters related to the presence of hemoglobin (e.g., oxygen saturation of hemoglobin, hemoglobin concentration levels). NIRS spectroscopy is based on the principle that light in the near-infrared range (700 to 1,000 nm) can pass easily through skin, bone and other tissues where it encounters hemoglobin located mainly within micro-circulation passages (e.g., capillaries, arterioles, and venules). Hemoglobin exposed to light in the near infra-red range has specific absorption spectra that varies depending on its oxidation state. Oxyhemoglobin (HbO₂) and deoxyhemoglobin (Hb) each act as a distinct chromophore. By using light sources that transmit near-infrared light at specific different wavelengths, and measuring differential changes in absorption, concentration changes of the oxyhemoglobin (HbO₂) and deoxyhemoglobin (Hb) within tissue can be monitored, and tissue oxygen saturation values (StO₂) can be derived. U.S. Pat. Nos. 6,456,862; 7,072,701; 8,078,250, and 9,364,175 describe NIRS spectroscopy devices and methods, each of which patent is hereby incorporated by reference in its entirety.

NIRS tissue oximeters can provide a non-invasively determined total hemoglobin value for a subject's tissue. As will be described herein, the total hemoglobin of tissue is proportional to relative blood volume within the sensed tissue (which volume may change over time). Using an optical based sensor placed on a tissue surface of a subject, a NIRS tissue oximeter can be used to interrogate tissue with different wavelengths of light (e.g., emit light into and detect light emanating from the tissue), and then process the detected light to calculate a total hemoglobin value for the tissue, and if desired also a tissue oxygen saturation (StO₂) value. For example, a sensor portion of a NIRS tissue oximeter placed on the forehead of a subject may be used to spectrophotometrically interrogate a subject's brain tissue and thereafter determine total hemoglobin and tissue oxygen saturation (StO₂) values for the subject's brain tissue.

Historically, circulatory blood hemoglobin values (i.e., a hemoglobin value representative of hemoglobin within circulatory blood) have been determined using an invasively extracted blood specimen. This specimen may be analyzed using a CO-oximeter or a hematology analyzer. A CO-oximeter is a spectrophotometric device that may be operated to also measure one or more types of hemoglobin present within a blood specimen; e.g., HbO₂, Hb, carboxyhemoglobin (COHb), methemoglobin (MetHb), by measuring the absorption of light at specific wavelengths passing through the blood specimen. The relative amounts of absorption at the different wavelengths enable a measurement of the respective types of hemoglobin present within the blood specimen. Hematology analyzers exist in many forms depending on the environment of use, e.g., automated and manual, reagent-based and broad spectrum photometric. Both methodologies are accepted as a reference for other technologies claiming a measurement of hemoglobin concentration in blood (i.e., volumetric value).

A primary difference between a prior art NIRS tissue oximeter and a CO-oximeter or a hematology analyzer is that the NIRS tissue oximeter is configured to determine a parameter value (e.g., hemoglobin, oxygen saturation) within tissue, whereas the CO-oximeter or hematology analyzer is configured to determine the same parameter value from a specimen of circulatory blood (i.e., an invasively collected specimen from an artery, vein, or lanced capillary bed). Using total hemoglobin as an example parameter, the total hemoglobin value determined within tissue using a prior art NIRS tissue oximeter can be affected by various different physiological parameters such as circulatory blood hemoglobin, hemoglobin concentration per volume of tissue, vasoreactivity, cardiac output, blood flow, partial pressure of carbon dioxide in arterial blood (PaCO₂), heart rate, blood volume, hematomas, and hyperemia. A total hemoglobin value derived from a circulatory blood specimen as determined using a CO-oximeter or a hematology analyzer will not be affected by these physiological parameters but requires invasive collection and specimen handling, which includes providing the specimen for analyzer throughput. In addition, invasive sampling of blood for analytic purposes is typically performed periodically; e.g., the blood is sampled and subsequently analyzed to derive a singular value. Hence, the information available from the blood analyzer approach is periodic, not continuous, and is retrospective. In contrast, continuous hemoglobin monitoring via NIRS, can provide an enhanced ability to identify current status blood constituents rise or fall trends, and a concomitant ability to address a trend if necessary. In addition, stable trending of a blood constituent such as hemoglobin can provide continuous information indicative of a normal state which can provide reassurance to a clinician.

What is needed is a method and apparatus operable to noninvasively determine blood hemoglobin parameters (e.g., total hemoglobin, differential concentrations of oxygenated hemoglobin) that improves current noninvasive technologies for continuously determining blood hemoglobin parameters.

### SUMMARY

According to an aspect of the present disclosure, a method of non-invasively determining continuous total hemoglobin data is provided. The method includes: a) using a near infra-red spectrophotometric (NIRS) sensing device on a continuous basis to sense a subject's tissue, wherein NIRS signals are produced from the sensing and are representative of total hemoglobin (THb); b) providing a body mass index (BMI) value of the subject; c) selectively calibrating the NIRS sensing device using a reference absolute THb value acquired from the subject, wherein the selective calibration includes calibrating or not calibrating the NIRS sensing device based on the BMI value; and d) determining continuous THb data using the produced NIRS signals.

In any of the aspects or embodiments described above and herein, the continuous THb data may be continuous relative THb data.

In any of the aspects or embodiments described above and herein, the continuous THb data may be continuous absolute THb data.

In any of the aspects or embodiments described above and herein, the method may include using the BMI value to determine a blood volume of the subject.

In any of the aspects or embodiments described above and herein, the step of selectively calibrating the NIRS sensing device may include using the BMI value to determine a blood volume of the subject and the subject blood volume is used in the calibrating or not calibrating the NIRS sensing device.

In any of the aspects or embodiments described above and herein, the method may include determining if a cardiopulmonary bypass (CPB) on the subject has occurred.

In any of the aspects or embodiments described above and herein, the step of determining if CPB on the subject has occurred utilizes a signal from a blood pressure measurement device.

In any of the aspects or embodiments described above and herein, the signal from the blood pressure measurement device may be a pulsatile waveform.

In any of the aspects or embodiments described above and herein, if the occurrence of a CPB is determined, the step of selectively calibrating the NIRS sensing device may include using the BMI value to determine a blood volume of the subject and the subject blood volume may be used in the calibrating or not calibrating the NIRS sensing device.

According to another aspect of the present disclosure, a method of non-invasively determining continuous total hemoglobin data during a medical procedure that includes cardiopulmonary bypass (CPB) performed using a CPB device. The method includes: a) using a near infra-red spectrophotometric (NIRS) sensing device on a continuous basis to sense a subject's tissue, wherein NIRS signals are produced from the sensing and are representative of total hemoglobin (THb); b) providing a body mass index (BMI) value of the subject; c) providing a priming volume value for the CPB device; d) determining an occurrence of the CPB; e) if the step of determining the occurrence of the CPB determines CPB has occurred, selectively calibrating the NIRS sensing device using a reference absolute THb value acquired from the subject, wherein the selectively calibrating includes a determination to calibrate or not calibrate the NIRS sensing device and the determination to calibrate or not calibrate uses the BMI value and the priming volume value; and f) determining continuous THb data using the produced NIRS signals.

In any of the aspects or embodiments described above and herein, the method may include using the BMI value to determine a blood volume of the subject value, and the blood volume may be used in the determination to calibrate or not calibrate.

According to another aspect of the present disclosure, a system for determining continuous total hemoglobin data from a subject is provided. The system includes a near infra-red spectroscopy (NIRS) sensing device and a controller. The NIRS sensing device is configured to sense a tissue region of the subject, and to produce NIRS signals from the sensing. The controller is in communication with the NIRS sensing device. The controller includes at least one processor and a memory device configured to store instructions, which instructions when executed cause the controller to: a) control the NIRS sensing device to sense a subject's tissue on a continuous basis, and produce NIRS signals from the sensing that are representative of total hemoglobin (THb); b) determine a blood volume value for the subject based on an input body mass index (BMI) value for the subject; c) selectively calibrating the NIRS sensing device using a reference absolute THb value acquired from the subject, wherein the selective calibration includes calibrating or not calibrating the NIRS sensing device based on the blood volume value; and d) determine continuous THb data using the produced NIRS signals.

In any of the aspects or embodiments described above and herein, the instructions when executed may cause the controller to determine if a cardiopulmonary bypass (CPB) on the subject has occurred.

In any of the aspects or embodiments described above and herein, the controller may be configured to communicate with a blood pressure measurement device and the determination of whether a CPB on the subject has occurred is based on signals from the blood pressure measurement device.

According to another aspect of the present disclosure, a non-transitory computer readable medium comprising software code sections which are adapted to perform a method for non-invasively determining continuous total hemoglobin data is provided. The method includes the steps of: a) controlling a near infra-red spectrophotometric (NIRS) sensing device on a continuous basis to sense a subject's tissue, the sensing producing NIRS signals that are representative of total hemoglobin (THb); b) determining a blood volume value of the subject using a body mass index (BMI) value; c) determining if the subject is undergoing cardiopulmonary bypass (CPB); d) if the subject is undergoing CPB, selectively calibrating the NIRS sensing device using a reference absolute THb value acquired from the subject, wherein the selectively calibrating includes a determination to calibrate or not calibrate the NIRS sensing device and the determination to calibrate or not calibrate uses the determined blood volume value; and e) determining the continuous THb data using the produced NIRS signals.

According to another aspect of the present disclosure, a method of monitoring a patient blood transfusion is provided. The method includes: a) providing a body mass index (BMI) value of the patient; b) using the BMI value to determine the patient's circulatory blood volume; c) using a near infra-red spectrophotometric (NIRS) sensing device on a continuous basis to sense the patient's tissue during the transfusion, wherein NIRS signals are produced from the sensing and are representative of a hemoglobin value of the subject's circulatory blood; and d) using the BMI value and the NIRS signals in the monitoring of the transfusion.

In any of the aspects or embodiments described above and herein, the hemoglobin value may be continuous total hemoglobin (THb) data.

According to another aspect of the present disclosure, a patient blood transfusion monitoring system is provided that includes a near infra-red spectroscopy (NIRS) sensing device and a controller. The NIRS sensing device is configured to sense a tissue region of the patient, and to produce NIRS signals from the sensing. The controller is in communication with the NIRS sensing device. The controller includes at least one processor and a memory device configured to store instructions, which instructions when executed cause the controller to: a0 control the NIRS sensing device to sense a patient's tissue on a continuous basis and produce NIRS signals from the sensing that are representative of a hemoglobin value of the patient's circulatory blood; b) determine a circulatory blood volume value for the patient using a provided body mass index (BMI) value of the patient; and c) provide information representative of the transfusion on a continuous basis using the NIRS signals and the determined circulatory blood volume.

According to another aspect of the present disclosure, a non-transitory computer readable medium comprising software code sections which are adapted to perform a method for monitoring a patient blood transfusion is provided. The method includes: a) determining a patient's circulatory blood volume using a provided body mass index (BMI) value of the patient; b) controlling a near infra-red spectrophotometric (NIRS) sensing device on a continuous basis to sense the patient's tissue during the transfusion, wherein NIRS signals are produced from the sensing and are representative of a hemoglobin value of the subject's circulatory blood; and c) providing information representative of the transfusion on a continuous basis using the NIRS signals and the determined circulatory blood volume.

The foregoing features and elements may be combined in various combinations without exclusivity, unless expressly indicated otherwise. These features and elements as well as the operation thereof will become more apparent in light of the following description and the accompanying drawings. It should be understood, however, the following description and drawings are intended to be exemplary in nature and non-limiting.

### BRIEF DESCRIPTION OF THE DRAWINGS

FIG. 1 is a diagrammatic representation of a NIRS sensing device with sensing transducers applied to a subject's head.

FIG. 2 is a diagrammatic representation of a NIRS sensing device transducer applied to a subject's head.

FIG. 3 is a diagrammatic planar representation of a NIRS sensing device transducer.

FIG. 4 shows a first graph of relative tissue hemoglobin values (ΔctHb) as a function of time, and a second graph of blood specimen total hemoglobin (tHb) and continuous total hemoglobin (THb) as a function of time, with each graph displaying respective values as a function of the same period of time.

FIG. 5 shows a graph of absolute blood total hemoglobin (THb) as a function of time with an initial calibration.

FIG. 5A shows a graph of relative blood total hemoglobin (ATHb) as a function of time without an initial calibration.

FIG. 6 shows a graph of relative tissue hemoglobin values (ΔctHb) as a function of time, indicating a two minute window where data has been flagged.

FIG. 7 shows a first graph of relative tissue hemoglobin (ΔctHb) as a function of time, and a second graph of tissue oxygen saturation (StO₂) as a function of time, with each graph displaying respective values as a function of the same period of time.

FIG. 8 shows a first graph of relative tissue hemoglobin (ΔctHb) as a function of time, and a second graph of tissue oxygen saturation (StO₂) as a function of time, with each graph displaying respective values as a function of the same period of time.

FIG. 9A shows a graph of relative tissue hemoglobin (ΔctHb) as a function of time, displaying ΔctHb and reference variable “R” values for a single evaluation period.

FIG. 9B shows a graph of relative tissue hemoglobin (ΔctHb) as a function of time, displaying ΔctHb and reference variable “R” values for multiple evaluation periods.

FIG. 9C shows a graph of relative tissue hemoglobin (ΔctHb) as a function of time, displaying ΔctHb and reference variable “R” values for multiple evaluation periods and a recalibration flag.

FIG. 10 shows a THb versus time graph disposed above a ΔctHb versus time graph (same time period) to illustrate calibrate/no calibrate.

FIG. 11 is a diagrammatic flow chart illustrating an embodiment of the present disclosure.

### DETAILED DESCRIPTION

The present disclosure is directed to a near infrared spectrophotometric (NIRS) system 20 and method for noninvasively measuring circulatory hemoglobin using a near infrared spectrophotometric (NIRS) sensing device 22, including logic (i.e., stored instructions) to determine whether calibration is appropriate for such a system 20, when a calibration may be performed, techniques for performing such calibration, and other functionalities. In some embodiments, the present disclosure system 20 may be independent of and in communication with the NIRS sensing device 22. For example, the present disclosure system 20 may be configured to use a NIRS sensing device 22 that is independently operable and configured to operate as a NIRS tissue oximeter independently. In these embodiments, the present disclosure system 20 may be configured to input and receive signal data from the NIRS sensing device 22 and process the signal data according to the functionality described herein. In other present disclosure embodiments, the present system 20 and the NIRS sensing device 22 may be integral with one another.

The NIRS sensing device 22 includes one or more transducers 24 and a system module that typically includes a display and a system controller 40 as will be detailed herein. Each transducer is capable of being operated to transmit light signals into the tissue of a subject and to sense for the transmitted light signals once they have passed through the subject's tissue via transmittance or reflectance. A variety of NIRS sensing device types can be modified according to aspects of the present disclosure, and the present disclosure is not therefore limited to any particular type of NIRS sensing device.

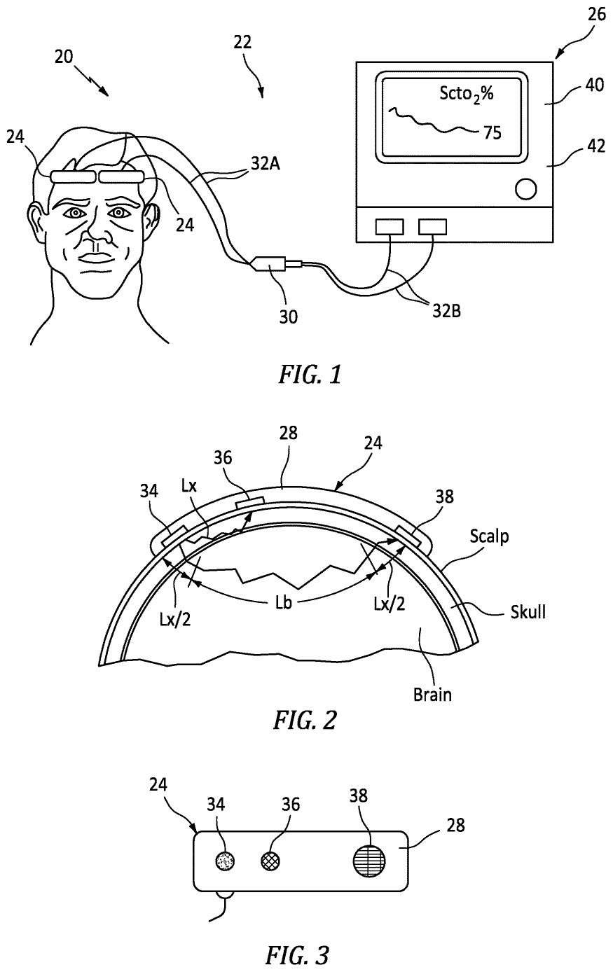

Referring to FIG. 1, a NIRS sensing device 22 is diagrammatically shown configured for sensing cerebral tissue. The present disclosure is not, however, limited to cerebral tissue applications. The NIRS sensing device 22 includes a system module 26 in communication with a pair of transducers 24 configured for attachment to a subject; e.g., on the subject's forehead. FIG. 2 diagrammatically illustrates one of the transducers 24 applied to a skull. FIG. 3 diagrammatically illustrates a transducer 24 embodiment in a planar view. The transducer 24 includes a transducer body 28 and may include a cable connector 30. A first connector cable 32A extends between the transducer body 28 and the cable connector 30. One or more second connector cables 32B extend between the cable connector 30 and the system module 26. In alternative embodiments, the cable connector 30 may be eliminated (e.g., one or more cables go directly from the transducer 24 to the system module 26), or the transducer 24 may be in communication with the system module 26 via wireless means. The transducer body 28 is typically a flexible structure that can be attached directly to a subject's body and includes one or more light sources and one or more light detectors. The transducer 24 embodiments shown in FIGS. 2 and 3 include a light source 34, a near light detector 36, and a far light detector 38, where the terms “near” and “far” indicate the relative distances from the light source 34. A disposable adhesive envelope or pad may be used to mount the transducer body 28 easily and securely to the subject's skin. The light source 34 may include, but is not limited to, light emitting diodes (“LEDs”) that emit light at a narrow spectral bandwidth at predetermined wavelengths. The light detectors 36, 38 may each include one or more photodiodes, or other light detecting devices. Non-limiting examples of acceptable NIRS sensing device transducers 24 are described in U.S. Pat. Nos. 9,988,873 and 8,428,674, both of which are commonly assigned to the assignee of the present application and both of which are hereby incorporated by reference in their entirety. NIRS sensing devices 22 and NIRS sensing device transducers 24 that may be used with the present disclosure include those configured for sensing from a skin surface of patient and those that are configured for sensing from other tissue surfaces of a patient such as an organ tissue surface; e.g., see U.S. Pat. No. 9,364,175 incorporated by reference above.

The system controller 40 may include any type of computing device, computational circuit, or any type of process or processing circuit capable of executing a series of instructions that are stored in a memory device 42. The system controller 40 may include multiple processors and/or multicore CPUs and may include any type of processor, such as a microprocessor, digital signal processor, co-processors, a micro-controller, a microcomputer, a central processing unit, a field programmable gate array, a programmable logic device, a state machine, logic circuitry, analog circuitry, digital circuitry, or the like, and any combination thereof. The instructions stored in memory may represent one or more algorithms for controlling the system 20, and the stored instructions are not limited to any particular form (e.g., program files, system data, buffers, drivers, utilities, system programs) provided they can be executed by the system controller 40. The instructions are configured to perform the methods and functions described herein. The system controller 40 may be configured (e.g., via electrical circuitry) to process various received signals (e.g., received from the transducers 24) and may be configured to produce certain signals to the same; e.g., signals configured to control operation of the transducers 24.

The memory device 42 may be a machine readable storage medium configured to store instructions that, when executed by one or more processors, cause the one or more processors to perform or cause the performance of certain functions. The memory device 42 may be a single memory device or a plurality of memory devices. A memory device 42 may be a non-transitory device and may include a storage area network, network attached storage, as well as a disk drive, a read-only memory, random access memory, volatile memory, non-volatile memory, static memory, dynamic memory, flash memory, cache memory, and/or any device that stores digital information. One skilled in the art will appreciate, based on a review of this disclosure, that the implementation of the system controller 40 may be achieved via the use of hardware, software, firmware, or any combination thereof.

In some embodiments, the present disclosure system 20 may include one or more input devices and one or more output devices. Non-limiting examples of an input device include a keyboard, a touchpad, or other device wherein a user may input data, commands, or signal information, or a port configured for communication with an external input device via hardwire or wireless connection, etc. Non-limiting examples of an output device include any type of display (e.g., as shown in FIG. 1), printer, or other device configured to display or communicate information or data produced by the system 20. The system 20 may be configured for connection with an input device or an output device via a hardwire connection or a wireless connection.

The system controller 40 (or other controller within the system 20) may be adapted to determine blood parameter values, including oxygen saturation values (that may be referred to as “SnO₂”, “StO₂”, “SctO₂”, “CrSO₂”, “rSO₂”, etc.) and hemoglobin concentration values (e.g., HbO₂, Hb, THb). U.S. Pat. Nos. 6,456,862; 7,072,701; 8,396,526; 8,923,943; 9,456,773; and 10,117,610, and PCT Publication No. WO 2018/187510 (each of which is hereby incorporated by reference in its entirety) each disclose methods for spectrophotometric blood parameter monitoring. The methods of determining blood parameters disclosed in U.S. Pat. Nos. 6,456,862 and 7,072,701 represent acceptable examples of determining a subject-independent NIRS tissue blood parameter values. The methods disclosed in U.S. Pat. Nos. 8,396,526; 8,923,943; 9,456,773; and 10,117,610 represent acceptable examples of a method of determining a NIRS tissue blood parameter value that accounts for the specific physical characteristics of the particular subject's tissue being sensed; i.e., a method that builds upon a subject-independent algorithm such as those disclosed in U.S. Pat. Nos. 6,456,862 and 7,072,701 to make it subject-dependent. PCT Patent Publication No. WO 2018/187510 discloses a method and system for noninvasively measuring circulatory hemoglobin and U.S. Provisional Patent Application No. 63/218,684 discloses a method and system for noninvasively measuring circulatory hemoglobin that accounts for hemodynamic confounders-both commonly assigned to the applicant of the present application. U.S. Provisional Patent Application No. 63/218,684 is hereby incorporated by reference in its entirety. Aspects of the present disclosure may include, but are not limited to, the methods described in the above identified patents and applications. The present disclosure described herein provides methods and techniques for modifying such methods, or for use with other NIRS methodologies, to enable a determination of a noninvasive NIRS circulatory THb value. Embodiments of the present disclosure may provide significant additional utility to the methods and systems disclosed in the above referenced patents and patent applications as well as to other methods and systems for noninvasively measuring circulatory hemoglobin. Hence, the present disclosure is not limited to use with the methods and systems disclosed in the above referenced patents and patent applications. In addition, as stated above the present disclosure contemplates that the present disclosure system 20 may be independent of and in communication with the NIRS sensing device 22 or integral with the NIRS sensing device 22. Hence, aspects of the functionality described herein may be performed in the present system 20 independently of the NIRS sensing device 22 or integrally within the NIRS sensing device 22, or any combination thereof.

Aspects of the present disclosure are directed to methods and systems for noninvasively measuring circulatory hemoglobin using a NIRS sensing device 22, including logic to determine the presence of a hemodynamic instability and/or hemodynamic changes in the subject's tissue. The presence of a hemodynamic instability and/or change may affect the accuracy of a measurement of circulatory hemoglobin produced using a NIRS sensing device. Hemodynamic changes may occur slowly or quickly over time. The present disclosure provides methodologies and system embodiments that facilitate non-invasive measurements of circulatory hemoglobin parameters with improved accuracy. Aspects of the present disclosure include logic/techniques for determining if calibration or recalibration of the NIRS system is appropriate (e.g., in view of hemodynamic instability and/or changes), when calibration/recalibration is appropriate, and techniques for performing such calibration. Aspects of the present disclosure further include methodologies for estimating BVF using NIRS data and blood gas data.

The NIRS sensing device 22 may be used to non-invasively determine a hemoglobin value for a subject's tissue (e.g., a relative tissue hemoglobin value, referred to hereinafter as “ΔctHb”) on a continuous basis. The term “continuously” as used herein (to describe a NIRS sensing device 22 continuously sensing) may be a NIRS sensing device 22 that senses and collects subject data on a periodic basis during a monitoring time period, which periodic basis is sufficiently frequent that it may be considered to be clinically continuous. The term “relative tissue hemoglobin value” is used herein to refer to changes in tissue hemoglobin between points in time; e.g., t1, t2 . . . tn. Various methodologies are known and may be employed by a NIRS sensing device 22 to determine a relative tissue hemoglobin value. The patents and patent applications referenced above provide examples of methodologies that may be used, but the present disclosure is not limited thereto.

The relative tissue hemoglobin can, in turn, be used to determine a continuous relative blood total hemoglobin (ΔTHb). A nonlimiting example of how relative tissue hemoglobin (ΔctHb) can be used to determine relative blood total hemoglobin (ΔTHb) is shown below in Equation 1:

$$Δ THb = Δ ctHb * \frac{Hb molar mass}{Local Blood Volume Fraction} \qquad ( Eqn . 1 )$$

Equation 1 may be expressed alternatively as shown in Equation 1A to illustrate relative blood total hemoglobin as a function of time.

$$Δ {THb}_{t} = ( Δ {ctHb}_{t} - Δ {ctHb}_{t 0} ) * \frac{Hb molar mass}{Local Blood Volume Fraction} \qquad ( Eqn . 1 A )$$

The term “local blood volume fraction” refers to the blood volume fraction (BVF) in the tissue sensed by the NIRS sensing device 22. BVF may vary as a function of the subject (e.g., inter-patient variability; different subjects, different BVFs) and may vary as a function of time (e.g., intra-patient variability). Regarding the latter, a subject's BVF can vary over time as a function of various physiologic conditions including but not limited to vaso-constriction/-dilation, venous congestion and the like. In some embodiments of the present disclosure, a local BVF may be estimated using sensed data (i.e., stored empirical data representing a clinically sufficient amount of data) produced noninvasively by the NIRS sensing device 22 and blood hemoglobin data. The empirical blood hemoglobin data may be determined using a hematology analyzer or a CO— oximeter to analyze invasively collected blood specimens, but the present disclosure is not limited to blood hemoglobin data produced from invasively collected blood specimens.

In some embodiments, artificial intelligence (AI)/machine learning (ML) techniques, algorithmic techniques, or the like may be utilized with empirical blood hemoglobin data to correlate NIRS relative tissue hemoglobin (ΔctHb) and blood circulatory THb; e.g., to determine an estimated BVF. As will be described herein, such a correlation may be used to obviate the need for an initial calibration using a blood circulatory THb value. In some embodiments, the correlation may take the form of a calibration parameter (“k”). A non-limiting example of how such a calibration parameter may be determined includes organizing (e.g., plotting) blood circulatory THb data versus NIRS total tissue hemoglobin values for analysis. A trend line can be determined (e.g., using a linear regression technique) from the plotted data points that represents a best fit to the data points. The trend line has a slope value and an intercept value, and the slope and intercept values may be used to determine a calibration parameter. The embodiment of determining a calibration parameter from plotted ΔctHb and blood circulatory THb values is used herein to illustrate how a calibration parameter may be determined, and the present disclosure is not limited thereto. As stated above, various techniques may be used with empirical data points to determine a calibration parameter. The following expressions are examples of how such a calibration parameter may be used to determine relative blood total hemoglobin (ΔTHb) using relative tissue hemoglobin (ΔctHb):

$$Δ THb = Δ ctHb * k \qquad ( Eqn . 2 )$$

$$Δ {THb}_{t} = ( Δ {ctHb}_{c} - Δ {ctHb}_{t 0} ) * k \qquad ( Eqn . 2 A )$$

PCT Publication No. WO 2018/187510, which is disclosed and incorporated by reference above, provides examples of how empirical data may be used to determine a calibration parameter.

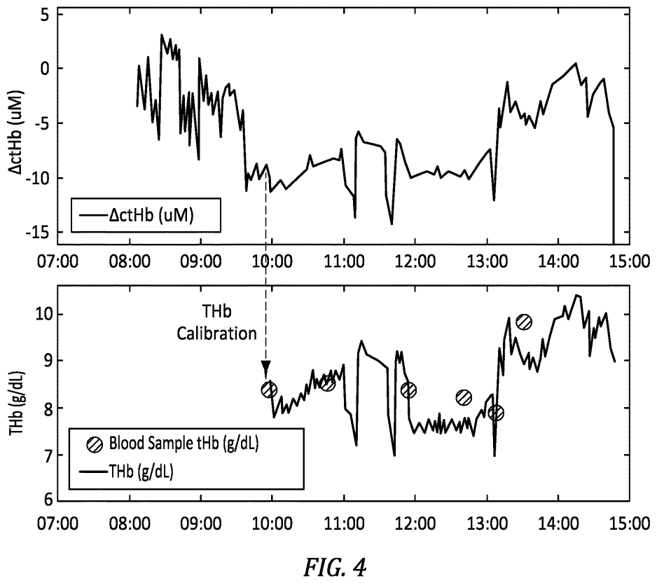

Once continuous relative blood total hemoglobin (ΔTHb) is determined, an absolute total hemoglobin (THb) may be determined using a total blood hemoglobin value determined from an invasively collected blood specimen. FIG. 4 includes a first graph of relative tissue hemoglobin (ΔctHb-μ moles) sensed as a function of time and a second graph of absolute blood total hemoglobin (THb) determined as a function of time based on relative tissue hemoglobin (ΔctHb) (alternatively continuous relative blood total hemoglobin (ΔTHb) determinable from ΔctHb may be used). In this example, the absolute total hemoglobin (THb) is initially calibrated (at about the 10:00 min mark) using a total blood hemoglobin value determined from an invasively collected blood specimen as indicated by the dot at the start of the data plot. The conversion of relative blood total hemoglobin (ΔTHb) to absolute blood total hemoglobin may be determined using Equation 3:

$$THb ( t ) = Δ THb + THb ( t 0 ) \qquad ( Eqn . 3 )$$

where THb(t0) is the total blood hemoglobin (THb) at a calibration point such as that shown in FIG. 4 and ΔTHb is determined as described above. The above described methodology provides a means for providing continuous absolute blood total hemoglobin information predominantly noninvasively, with minimal invasive blood collections required.

In the determination of total hemoglobin (relative or absolute) some system embodiments may take into consideration a variety of factors (referred to herein as “oximetry features”). These oximetry features may relate to physiological features of a subject (e.g., StO₂ determined in a singular frequency band or multiple frequency bands, tissue perfusion index or “TPI”, ΔctHb, skin temperature), or intermediate features (e.g., tissue optical properties or “TOP”, or analytically determined constants reflecting individual subject characteristics—e.g., Cₙ*StO₂), or NIRS oximetry features (e.g., length of the path travelled by photons between a transducer light source and light detector, optical densities, gains), or statistical features (e.g., average values, mean values, median values, standard deviations, of different data windows and the like), or the like, including any combination thereof. Examples of tissue optical properties or “TOPs” include skin pigmentation, muscle and bone density. These oximetry features may be accounted for in an expression for blood total hemoglobin (absolute or relative). A non-limiting example of how oximetry features may be accounted for in an expression for absolute blood total hemoglobin (THb) is shown in Equation 4 below.

$${THb}_{t} = BG ( t 0 ) + ( Δ ctHb ( t ) - Δ ctHb ( {t}_{0} ) ) * k + ( {C}_{o} + {\sum }_{n = 1}^{5} {C}_{n} * {f}_{n , t} ) \qquad ( Eqn . 4 )$$

Where “BG(t0)” is a total blood hemoglobin value determined from an invasively collected blood specimen (e.g., blood gas), “k” is a calibration parameter (as described herein), “C₀” is a constant, “f” is an oximetry feature, “Cn” is a derived constant for each oximetry feature, and the summation indicates one through five oximetry features being considered.

Alternatively, in some embodiments continuous absolute blood total hemoglobin information may be provided without using an initial total blood hemoglobin value determined from an invasively collected blood specimen for calibration purposes.

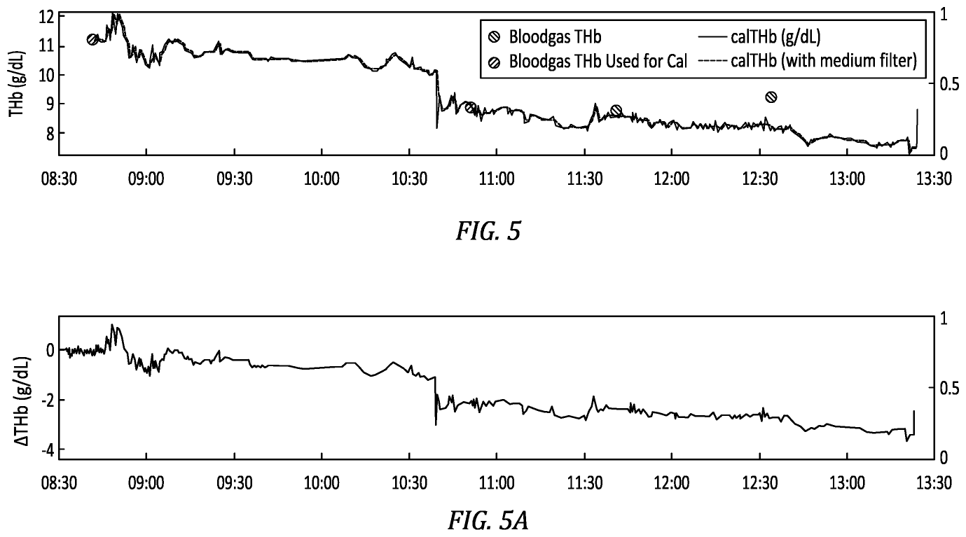

FIG. 5 is a graph of continuous absolute blood total hemoglobin (THb), shown in units of grams per deciliter (g/dL) as a function of time. The g/dL scale (on Y-axis) shown in FIG. 5 is from about 8 g/dL to about 12 g/dL. Similar to FIG. 4, the THb data shown in FIG. 5 is initially calibrated using a total blood hemoglobin value determined from an invasively collected blood specimen. The calibrated THb data shown in FIG. 5 begins at about the 8:40 minute point with the dot indicating a calibration. The data shown in FIG. 5 also indicates other invasively collected total blood hemoglobin values at about the 10:50 minute point, at about the 11:40 minute point, and at about the 12:35 minute point. The blood hemoglobin values at about the 10:50 minute point is also used for calibration. The THb data values vary from just under 8 g/dL to about 12 g/dL. The THb data shown in FIG. 5 may be produced using an expression like that shown in Equation 4 above.

FIG. 5A is a graph of relative blood total hemoglobin (ΔTHb), also shown in units of grams per deciliter (g/dL) as a function of time. The ΔTHb data shown in FIG. 5A is that shown in FIG. 5, this time produced without initial calibration. The g/dL scale (on Y-axis) shown in FIG. 5A is from about 0 g/dL to about −4 g/dL. The ΔTHb data values in FIG. 5A vary from just over 0 g/dL to about −4 g/dL. The ΔTHb data shown in FIG. 5A may be produced using a variation of the expression shown in Equation 4 above, shown below in Equation 4A.

$$Δ TH {b}_{t} = ( Δ ctHb ( t ) - Δ ctH b ( {t}_{0} ) ) * k + ( {C}_{o} + {\sum }_{n = 1}^{5} {C}_{n} * {f}_{n , t} ) \qquad ( Eqn . 4 A )$$

The THb and ΔTHb data shown in FIGS. 5 and 5A track with a high degree of agreement. A difference between the two is a shift in the THb values (i.e., about 8 to 12 g/dL in the calibrated data versus about −4 to 0 g/dL). Clearly, the data plot in FIG. 5A shows the same data trend as a function of time as the data plot in FIG. 5, but does not require invasive specimen data and therefore provides the relative THb data without the need for an initial calibration. The ability of embodiments of the present disclosure to provide such information non-invasively is understood to beneficial in many instances.

As stated above, the present disclosure includes calibration methodologies for ensuring the absolute blood total hemoglobin information produced is not erroneous or otherwise compromised, as well as methodologies (e.g., AI/machine learning based) for estimating BVF using NIRS data and blood gas data produced from an invasive measurement.

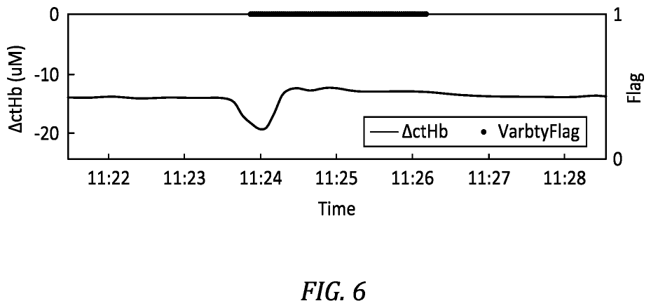

A first calibration methodology example is directed to indicating when it is appropriate to calibrate the NIRS sensing system to enable it to accurately provide noninvasive continuous absolute blood total hemoglobin information. Certain factors, when present, may affect the accuracy of a calibration. Hence, the system 20 may be configured to identify the presence or absence of such a factor, and if present then flag or prevent a user from performing the calibration. For example, under circumstances when a NIRS sensing device 22 produces relative tissue hemoglobin (ΔctHb) data as a function of time, the produced relative tissue hemoglobin (ΔctHb) data may be unstable; e.g., variable beyond a predetermined threshold range. Under those circumstances, the present disclosure system 20 may interpret that ΔctHb instability as an indication that the relative tissue hemoglobin data is suspect. FIG. 6 illustrates a graph of relative tissue hemoglobin (ΔctHb) data as a function of time. The methodology may, for example evaluate ΔctHb data within a rolling predetermined window; e.g., a two minute “evaluation” window. If the collected ΔctHb data varies in magnitude outside of the predetermined threshold range, then the system 20 may flag that variance to indicate that the ΔctHb data collected within the evaluation window should not be used for purposes of calibrating the NIRS sensing system for absolute blood total hemoglobin. FIG. 6 indicates that the two minute window between 11:24 and 11:26 is flagged.

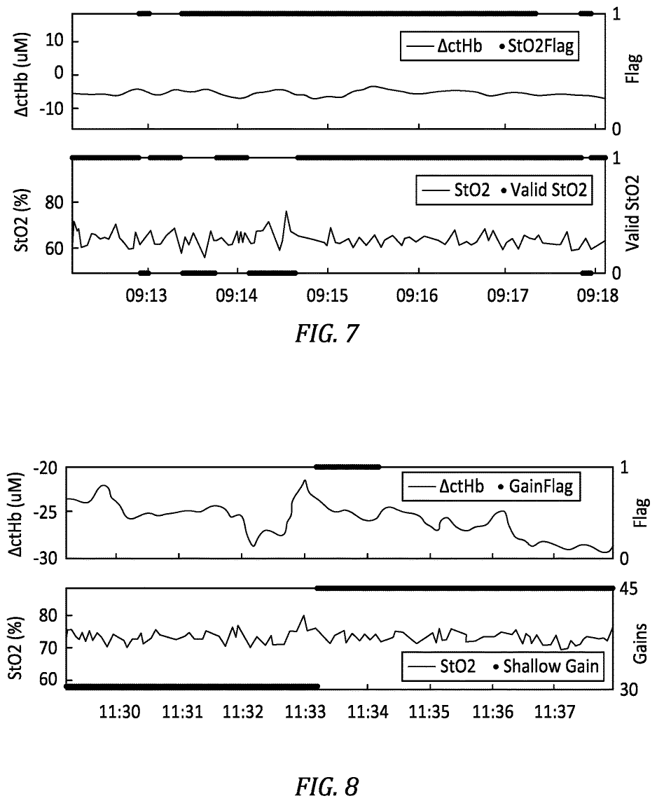

In a second calibration methodology example, the present disclosure may utilize the NIRS sensing device 22 to determine a tissue oxygen saturation value (StO₂). Tissue oxygen saturation values (StO₂) produced by the NIRS sensing device 22 may be used to evaluate whether a transducer 24 of the NIRS sensing device 22 is appropriately placed on the subject, or otherwise evaluate the operation of the transducer 24. FIG. 7 illustrates a ΔctHb versus time graph disposed above a StO₂ versus time graph (same time period). Both graphs include a “1” line disposed on an upper edge of the respective graph and a “0” line disposed on a base edge of the respective graph. The “1” line of the StO₂ versus time graph is an indicator that the NIRS sensing data (i.e., StO₂) is acceptable/valid, and the “0” line of the StO₂ versus time graph is an indicator that the NIRS sensing date (i.e., StO₂) is unacceptable/invalid. The “1” line of the ΔctHb versus time graph is an indicator that the ΔctHb data is not acceptable/valid for calibration. It should be noted that the StO₂ values may be based on raw signals having a signal quality and variability, and in some embodiments the acceptable/unacceptable character of the StO₂ data and the ΔctHb data may consider the signal quality and variability of the raw signals.

In some embodiments, the StO₂ sensing data may be further evaluated as a function of time. For example, StO₂ data may vary naturally (e.g., due to system issues, rapid transient changes in StO₂, signal quality/variability) from an acceptable value to an unacceptable value. These fluctuations may occur seldomly or frequently, and may vary in duration. The further evaluation may include evaluating the StO₂ fluctuations over an evaluation period. The further evaluation may consider the magnitude of the fluctuations and/or the collective duration of the fluctuations within the evaluation period. For example, the collective duration of unacceptable fluctuations within a given evaluation period may be continuously evaluated relative to a predetermined collective threshold (e.g., a percentage such as 10% of the evaluation window duration). If the collective duration of unacceptable fluctuations exceeds the collective threshold, then all of the StO₂ data collected to that point in the evaluation window may be deemed unacceptable and the corresponding ΔctHb data may be flagged as unacceptable for use in calibration. In some embodiments, once the collective threshold is reached, a new evaluation period may be initiated, and the collective duration of unacceptable fluctuations set to zero.

In a third calibration methodology example, a NIRS sensing device 22 may be used to determine a tissue oxygen saturation value (StO₂). FIG. 8 illustrates a ΔctHb versus time graph disposed above a StO₂ versus time graph (same time period). In this example, the parameters (ΔctHb and StO₂) are evaluated in terms of raw signal strength, where raw signal strength is depicted in the graphs via a proxy such as amplification gain by the system. Raw signal strength may be an indicator of changes in the tissue being sensed by the NIRS sensing device 22 (e.g., changes in blood volume within the tissue, changes in blood oxygen saturation within the tissue) and may be used to determine the acceptability of data for calibration purposes. Changes in the tissue can result from various events, such as hemodilution produced by a cardiopulmonary bypass pump, and the like. Raw signal strength changes relative to a predetermined threshold may be used to determine whether the NIRS sensing data (i.e., StO₂) is stable/acceptable or is unstable/unacceptable/invalid. In FIG. 8, the StO₂ versus time graph indicates raw signal strength (via amplification gain proxy) at a first level indicated at a value of “30” on an arbitrary scale for a time period between just prior to 11:30 to about 11:33. At about 11:33, the StO₂ versus time graph indicates raw signal strength (via amplification gain proxy) at a second level indicated at a value of “45” on an arbitrary scale for a time period between just at about 11:33 to beyond 11:37. In this example, the ΔctHb versus time graph (same period of time as the StO₂ versus time graph) indicates a no-calibration flag being raised (via the line disposed at the “1” line between at about 11:33 to about 11:34.

Some embodiments of the present disclosure may include a recalibration algorithm that is based on accumulated ΔctHb changes; e.g., another measure of ΔctHb stability/NIRS sensing device 22 performance. The accumulated ΔctHb changes may be based on accumulated ΔctHb variance data. The following is a nonlimiting example of a recalibration algorithm.

The algorithm may include a reference variable “R” that is initially (e.g., at the beginning of a THb monitoring period) at Step 1 set to:

$$R = Δ ctHb ( {t}_{0} ) \qquad ( Eqn . 5 )$$

For each new tissue specimen analysis, the algorithm accumulates ΔctHb deviation data for a second reference value “CumDev”, which was initially set to zero at Step 1. Equation 6 illustrates a nonlimiting example of how reference value CumDev may be populated in Step 2:

$$\vert Δ ctHb ( {t}_{n} ) - R \vert > 5 μM \to CumDev = CumDev + ( Δ ctHb ( {t}_{n} ) - R ) \qquad ( Eqn . 6 )$$

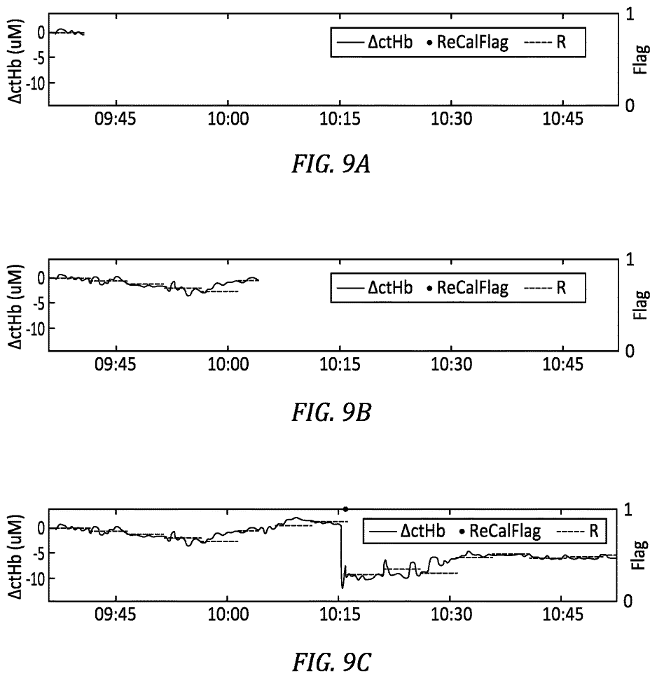

For each new tissue specimen analysis, the algorithm may include evaluating the reference value CumDev to determine whether the accumulated ΔctHb deviation data represented by CumDev exceeds a predetermined threshold. If the accumulated ΔctHb deviation data represented by reference value CumDev exceeds the predetermined threshold value, then a “recalibration” flag may be raised. If the recalibration flag is raised, the reference variable R may be reset as shown above in Equation 5 and the reference value CumDev may be set to zero when the recalibration flag is raised, and the process begins again as described. If the accumulated ΔctHb deviation data represented by reference value CumDev does not exceed the predetermined threshold value, then no recalibration flag is raised. After some predetermined period of time (e.g., an evaluation period of 5 minutes) without raising a “recalibration” flag, the reference variable R may be reset as shown above in Equation 5 and the reference value CumDev set to zero at the end of the then current evaluation period and the process may then be repeated. FIG. 9A shows a graph of ΔctHb versus time with ΔctHb data and an “R” value for the first five minute evaluation period, and FIG. 9B shows the same graph of ΔctHb versus time, now with ΔctHb data and an “R” value for each of a plurality of five minute evaluation periods. Neither FIG. 9A nor FIG. 9B show a recalibration flag. FIG. 9C shows the graph of ΔctHb versus time, showing ΔctHb data and “R” values for multiple five minute evaluation periods. At about 10:15, a recalibration flag is raised. Once the recalibration flag is raised, the reference variable R is reset and the reference value CumDev is set to zero and the process begins again. FIG. 9C illustrates ΔctHb data and “R” values for multiple five minute evaluation periods subsequent to the recalibration flag being raised. In this manner, embodiments of the present disclosure maintain a continuous evaluation of ΔctHb deviation. The above described methodology is an example of how ΔctHb deviation data may be monitored for purposes of identifying when recalibration may be warranted, and the present disclosure is not limited to this example.

Another nonlimiting example of a recalibration algorithm may utilize a statistical parameter based on ΔctHb values collected within an evaluation window occurring during a period of time prior to the then current point in time; e.g., ΔctHb values collected within the previous “X” minutes. For example, the ΔctHb data collected within the evaluation window may be processed to determine a median value. The then current ΔctHb value may be evaluated using the determined ΔctHb median value and a threshold value. For example, the evaluation may determine the absolute difference between the current ΔctHb value and the ΔctHb median value and compare that difference to the threshold value as shown in Equation 7 below:

$$\vert Δ {ctHb}_{c u r rent} - median Δ {ctHb}_{evaluation window} \vert > threshold value \qquad ( Eqn . 7 )$$

If the absolute difference between the current ΔctHb value and the ΔctHb median value exceeds the threshold value, then a “recalibration” flag may be raised.

In some embodiments, the above described algorithm may be configured to select a corrective action other than raising a “recalibration” flag, or a corrective action in addition to the “recalibration” flag. The present disclosure is not limited to the recalibration algorithm described above.

In some embodiments, the present disclosure may include a calibration algorithm that utilizes machine learning or other artificial intelligence technique. In these embodiments, a function “f” may be used to represent multivariate machine learning models using oximetry data. Equation 8 represents a generic function that represents the approach.

$$THb ( t ) = BG ( t 0 ) + f ( oximetry data ) \qquad ( Eqn . 8 )$$

The variable “BG” represents a THb value acquired using a technique such as a blood gas/hematology analyzer (or the like). The term “oximetry data” is defined above. A more specific example of an algorithm that may be used is shown in Equation 9:

$$THb ( t ) = BG ( t 0 ) + ( Δ ctH b ( t ) - Δ ctHb ( {t}_{0} ) ) * k \qquad ( Eqn . 9 )$$

The variable “ΔctHb (to)” represents relative tissue hemoglobin concentration at the time of a calibration; e.g., calibration as described above using blood gas THb. The variable “k” represents a correlation factor (described above) that may be computed with a linear regression technique using a machine learning training dataset. The machine learning training dataset contains a clinically significant amount of clinical data.

The algorithm utilized with machine learning may be developed in a variety of different ways. As an example, in a first step, a training dataset containing a clinically significant amount of clinical data may be split into a training dataset portion and one or more testing/validation dataset portions; e.g., a training dataset portion, a cross-validation dataset portion, and a final validation dataset portion.

A second step in the algorithm development process may involve selecting a training approach for developing a THb calculation model. A first example of a training approach that may be used is a direct approach that estimates the change in THb since the last device calibration; e.g., functionally expressed in Equation 10:

$$B {G}_{( t )} - B {G}_{( t 0 )} = f ( oximetry data ) \qquad ( Eqn . 10 )$$

Second and third examples of a training approach that may use utilize a boosting approach; e.g., an approach that estimates the error of the model expressed in Equation 9, expressed below in Equation 11:

$$B {G}_{( t )} - B {G}_{( t 0 )} - ( k * ( Δ {ctHb}_{( t )} - Δ {ctHb}_{( t 0 )} ) ) = f ( oximetry data ) \qquad ( Eqn . 11 )$$

or an approach that estimates the error of a multivariate model as expressed in Equation 12 below:

$$B {G}_{( t )} - B {G}_{( t 0 )} - modelX = f ( oximetry data ) \qquad ( Eqn . 12 )$$

where the variable modelX represents a multivariate model. The present disclosure is not limited to the above examples of training approaches.

As indicated above, the “oximetry data” may include a variety of different data types that may be considered in the development of the machine learning algorithm. AI/ML techniques may be used (e.g., correlation, linear regression, coherence, decision trees) to identify the oximetry data types most appropriate. Taking into consideration the identification of applicable oximetry data and the further identification of oximetry data most appropriate for machine learning, a more in depth algorithm expression may be used, such as that shown above in Equation 4.

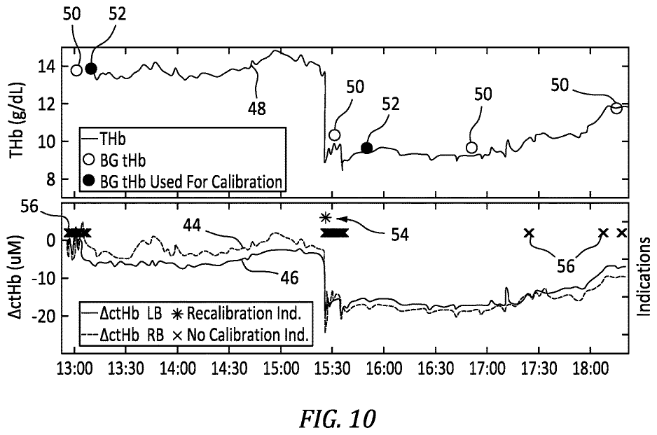

FIG. 10 illustrates a THb versus time graph disposed above a ΔctHb versus time graph (same time period) to illustrate calibrate/no calibrate. The ΔctHb versus time graph includes a first line 44 of continuous line depicting the “ΔctHb LB” (where “LB” refers to NIRS data acquired from the left hemisphere of a subject's brain), and a second line 46 depicting the “ΔctHb RB” (where “RB” refers to NIRS data acquired from the right hemisphere of a subject's brain). The ΔctHb versus time graph also includes markers “X” identifying a “no-calibration” indication and markers “*” identifying a recalibration indication. The THb versus time graph shown in FIG. 10 includes a line 48 depicting THb data, markers 50 indicating a blood gas (BG) derived THb value, and markers 52 indicating a blood gas (BG) derived THb value that is used for calibration. The data shown in the FIG. 10 graphs includes an initial noisy region at about 13:00 and a region of sharp instability at just before 15:30 (e.g., caused by a cardiopulmonary bypass, or the like). As indicated in the THb versus time graph, at just past 13:00 a blood gas (BG) derived THb value is provided (labeled as 50) that is not used for calibration due the instability of the ΔctHb data at that point in time. Shortly thereafter, another blood gas (BG) derived THb value is provided (labeled as 52) that is used for calibration. At just before 15:30 in the ΔctHb versus time graph, a recalibration flag 54 is indicated. At about 15:30 in the THb versus time graph, a blood gas (BG) derived THb value is provided (labeled as 50) that is not used for calibration due the instability of the ΔctHb data at that point in time. Shortly after 15:30, another blood gas (BG) derived THb value is provided (labeled as 52) that is used for re-calibration. Subsequent THb data shows good agreement with blood gas (BG) derived THb values (labeled as 50) after 16:30. The ΔctHb versus time graph includes symbols “X” (labeled as 56) used to indicate no calibration; i.e., pursuant to the present disclosure, the then current circumstances are such that no calibration should be performed.

Aspects of the present disclosure may be utilized to provide clinically useful medical information during surgical procedures that include cardiopulmonary bypass (CPB) and blood transfusions.

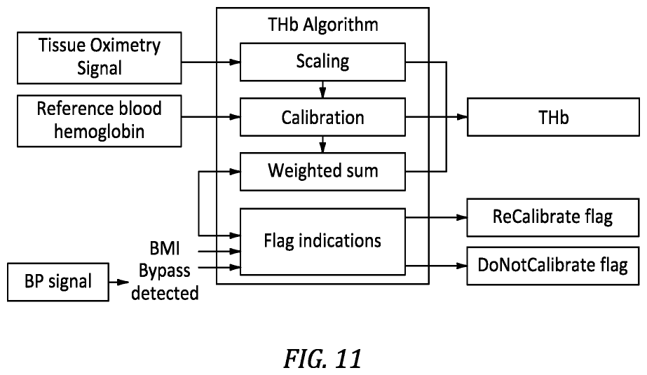

Referring to FIG. 11, CPB is a form of extracorporeal circulation that can be used to provide circulatory and respiratory support during heart surgery. A device used to perform CPB (i.e., a “CPB device”) typically includes pumps, cannulae, tubing, a reservoir, an oxygenator, a heat exchanger, and other elements. During use, the patient is connected to the CPB device to form an extracorporeal circuit with the CPB circulating fluid passing through the patient and the CPB device. The CPB circulating fluid includes both the patient's blood and a priming solution that may include crystalloids, or colloids, or some mixture thereof. The priming solution is known to hemodilute the patient's blood during which the patient's hemoglobin levels may suddenly drop to a low value. The total volume of the circulating fluid (TCV) during bypass may be defined as follows:

$$Total circulating volume ( TCV ) = subject ’ s blood volume + priming volume \qquad ( Eqn . 13 )$$

The amount of priming solution may differ depending on the device used to perform the CPB, but the amount of priming solution associated with a given CPB device is typically known. The subject's blood volume also differs with respect to patient's physical characteristics; e.g., an obese patient will have a greater blood volume value than a petite patient. The hemodilution will create a change in the subject's circulatory hemoglobin (regardless of the size of the subject) that is not attributable to any physiologic factor known to cause changes in circulatory hemoglobin. Hence, it would be advantageous to identify a change in the patient's circulatory hemoglobin that is attributable to CPB so that the change can be identified and correctly attributed.

Embodiments of the present disclosure are configured to identify a change in the subject's circulatory hemoglobin that may occur during a medical procedure that involves extracorporeal blood flow such as CPB. These embodiments of the present disclosure utilize the subject's body mass index (BMI) as a parameter. BMI is a value derived from the mass/weight and height of the subject. BMI may be defined as the weight of the subject divided by the square of the subject's body height, and is expressed in units of kg/m2.

$$BMI = \frac{subject weight ( kg )}{{( subject height ( m ) )}^{2}} \qquad ( Eqn . 14 )$$

The present disclosure may utilize the subject's BMI in a determination of the subject's circulatory blood volume (BV). The subject's BV may be determined in a variety of different ways. For example, numerous mathematical algorithms are known that provide a relationship between BV and BMI. An example of such a mathematical algorithm is the following Lemmens/Bernstein/Brodsky equation:

$$B V = \frac{70}{\sqrt{\frac{BMI}{22}}} \qquad ( Eqn . 15 )$$

The Lemmens/Bernstein/Brodsky equation is a non-limiting example of a mathematical algorithm that provides a relationship between BV and BMI for adult subjects. Other mathematical algorithms that provide a relationship between BV and BMI may be used alternatively. In some instances, different mathematical algorithms may be used for adult men and women and for children. Still other mathematical algorithms may utilize a pre-operative hematocrit value as well as different expressions for men, women, and children. The aforesaid one or more mathematical algorithms that provide a relationship between BV and BMI may form part of the executable stored instructions.

The present disclosure is not limited to using a mathematical algorithm that provides a relationship between BV and BMI. Some embodiments of the present disclosure may utilize a lookup table, a data table, database, or any stored data structure that provides a correlation between BMI values and BV values (collectively referred to herein after as a “data table”) to store correlated BV values and BMI values; e.g., a data structure wherein a given BMI can be identified and the correlated BV value can be determined. A BV/BMI data table may form part of the executable stored instructions. As will be explained below, the present disclosure may use a known priming solution volume (i.e., an input value) and a determined patient BV to evaluate whether a change in the patient's circulatory hemoglobin may be attributable to CPB or other factors.

During a surgical procedure the onset of CPB is determinable. Some embodiments of the present disclosure may utilize data attributable to a blood pressure measurement to determine the onset of CPB. For example, a subject's blood pressure may be monitored; e.g., either by the present disclosure system itself or by a blood pressure sensing device independent of the present disclosure system but in communication with the present system. The onset of CPB can be detected, for example, by the loss of a pulsatile waveform from the data signals produced by the blood pressure sensing device. Monitoring pulsatile waveform signals using a blood pressure device is a nonlimiting example of how the onset of CPB may be determined.

The circulatory fluid used in CPB may cause some level of hemodilution in the subject and therefore a decrease in the subject's circulatory hemoglobin. The data in FIG. 10 illustrates a sharp change in THb at just before 15:30 that is attributable to hemodilution associated with a CPB. Embodiments of the present disclosure provide a means to evaluate the aforesaid decrease in the subject's circulatory hemoglobin. For example, the present disclosure may utilize a determined BV for the subject (determined using BMI and the stored instructions) and a known priming solution volume to evaluate the decrease in the subject's circulatory hemoglobin. The aforesaid evaluation may include, for example, comparing the sensed decrease in the subject's circulatory hemoglobin to a decrease in circulatory hemoglobin that is calculated based on the determined BV/BMI and the known priming solution volume. The difference between the sensed decrease in the subject's circulatory hemoglobin and the calculated decrease in circulatory hemoglobin can be used to determine if the decrease is attributable solely to the CPB and the hemodilution attributable thereto, or whether another factor may be in play. If the decrease in circulatory hemoglobin is attributable to the hemodilution, then that suggests that the NIRS sensing device is operating appropriately. If the decrease in circulatory hemoglobin deviates from that attributable to the hemodilution, then that suggests that recalibration of the NIRS sensing device may be appropriate.

As indicated above, embodiments of the present disclosure includes logic (e.g., stored instructions) to determine whether calibration is appropriate for the present disclosure continuous and noninvasive system, when a calibration may be performed, and techniques for performing such calibration. In some embodiments, the aforesaid logic may include producing an indication that recalibration is appropriate; e.g., a significant change in ΔctHb values, may cause a “recalibration flag” to be raised. In an instance wherein a patient is subject to a surgical procedure that utilizes CPB, a notable change in the patient's circulatory hemoglobin will likely be sensed as a result of the CPB hemodilution. Absent a determination that CPB has begun, aspects of the present disclosure may give rise to the aforesaid indication that recalibration should occur. The above described embodiments, however, may evaluate whether a change in the patient's circulatory hemoglobin is attributable to CPB and if so, pause any indication that recalibration should occur. More generally, the above described present disclosure embodiments utilize BMI as a means to determine a subject's BV and utilize the same within a noninvasive continuous circulatory hemoglobin system and method and thereby improve the consistency and accuracy of the system and method. This is particularly true when noninvasive continuous circulatory hemoglobin is used in a medical procedure (e.g., CPB) where hemodilution may occur.

As indicated above, aspects of the present disclosure may provide information useful in the administration of a blood transfusion. Blood transfusions are common in a variety of different applications; e.g., to replace blood shed during a surgical procedure, or in the treatment of physiologic conditions such as anemia, cancer, hemophilia, kidney disease, liver disease, severe infections, sickle cell disease, and thrombocytopenia. In some instances, the transfusion may be an exchange transfusion that involves removing a volume of blood containing a diseased constituent and replacing it with a volume of blood (“donor blood”) that is free of the diseased constituent. The donor blood may be autologous blood or allogenic blood, and may include intravenous solutions that include crystalloids and/or colloids to maintain normovolemia. During a transfusion, the donor blood may have a lower hemoglobin concentration than the patient's blood and is also likely to contain non-blood diluents. As a result, hemodilution may occur. The following nonlimiting examples illustrate the utility of aspects of the present disclosure as they may be used with transfusion procedures.

Treatment for sickle cell disease may involve an exchange transfusion wherein the patient's blood containing diseased red blood cells (having a sickle-like shape caused by the disease affecting the hemoglobin of the red blood cell) are exchanged with donor blood containing healthy red blood cells. Aspects of the present disclosure permit a BV determination utilizing an input value for the patient's BMI. The patient's BV determination is useful in determining the amount of donor blood necessary to accomplish the desired degree of blood exchange and the degree to which a patient's BV has been exchanged via the transfusion. As stated above, the transfusion of donor blood will likely cause some level of hemodilution. The present disclosure permits the patient's hemoglobin to be noninvasively monitored during the transfusion process on a continuous basis. The aforesaid noninvasively acquired information can be used to assess whether a patient's hemoglobin concentration is within an acceptable range, or conversely whether the patient is trending towards anemia or polycythemia. The continuous, real-time capabilities of the present disclosure are understood to provide considerable clinical benefit in contrast to prior art practices in which a volume of donor blood is administered, and then after a period of time the patient's circulatory blood hemoglobin is reassessed.

Another example of the present disclosure's utility during a transfusion relates to polycythemia and Polycythemia vera (PV). Polycythemia can occur in newborns for a variety of different reasons. PV is a rare, chronic blood cancer in which a person's body makes too many red blood cells, white blood cells, and platelets. PV can occur in persons of any age however it is more common in older people. Too many red blood cells can cause a patient's blood to thicken to a point where blood flow into narrower capillaries is impeded or prevented. As a result, tissue associated with the capillaries may suffer from low oxygen saturation. Treatment for polycythemia may involve transfusing a patient with a low hemoglobin donor blood or intravenous solution for the purpose of decreasing the patient's abnormally high hemoglobin to an acceptable level. Aspects of the present disclosure permit a BV determination utilizing an input value for the patient's BMI. The patient's BV determination is useful in determining the amount of donor blood/intravenous solution necessary to accomplish the desired hemoglobin adjustment and the degree to which a patient's BV has been exchanged via the transfusion. The present disclosure also permits the patient's hemoglobin to be monitored during the transfusion process on a continuous basis. The continuous hemoglobin information can be used to assess in real-time whether a patient's hemoglobin is within an acceptable range, or too high or too low. These continuous, real-time capabilities are understood to provide considerable clinical benefit in contrast to prior art practices in which a volume of donor blood is administered, and then after a period of time the patient's circulatory blood hemoglobin is reassessed.

As indicated above, the functionality described herein may be implemented, for example, in hardware, software tangibly embodied in a computer-readable medium, firmware, or any combination thereof. In some embodiments, at least a portion of the functionality described herein may be implemented in one or more computer programs. Each such computer program may be implemented in a computer program product tangibly embodied in non-transitory signals in a machine-readable storage device for execution by a computer processor. Method steps of the present disclosure may be performed by a computer processor executing a program tangibly embodied on a computer-readable medium to perform functions of the present disclosure by operating on input and generating output. Each computer program within the scope of the present claims below may be implemented in any programming language, such as assembly language, machine language, a high-level procedural programming language, or an object-oriented programming language. The programming language may, for example, be a compiled or interpreted programming language.

While the invention has been described with reference to an exemplary embodiment(s), it will be understood by those skilled in the art that various changes may be made, and equivalents may be substituted for elements thereof without departing from the scope of the invention. For example, the term “total hemoglobin” is described herein as being the sum of HbO₂ and Hb. The present disclosure contemplates embodiments wherein a total hemoglobin value may include contributions from one or more other types of hemoglobin; e.g., carboxyhemoglobin (COHb), methemoglobin (MetHb). In addition, many modifications may be made to adapt a particular situation or material to the teachings of the invention without departing from the essential scope thereof. Therefore, it is intended that the invention is not limited to the particular embodiment(s) disclosed, but that the invention will include all embodiments falling within the scope of the appended claims

It is noted that the embodiments may be described as a process which is depicted as a flowchart, a flow diagram, a block diagram. Although, any one of these structures may describe the operations as a sequential process, many of the operations can be performed in parallel or concurrently. In addition, the order of the operations may be rearranged. A process may correspond to a method, a function, a procedure, a subroutine, a subprogram, or the like.

The singular forms “a,” “an,” and “the” refer to one or more than one, unless the context clearly dictates otherwise. For example, the term “comprising a specimen” includes single or plural specimens and is considered equivalent to the phrase “comprising at least one specimen.” The term “or” refers to a single element of stated alternative elements or a combination of two or more elements unless the context clearly indicates otherwise. As used herein, “comprises” means “includes.” Thus, “comprising A or B,” means “including A or B, or A and B,” without excluding additional elements.

It is noted that various connections are set forth between elements in the present description and drawings (the contents of which are included in this disclosure by way of reference). It is noted that these connections are general and, unless specified otherwise, may be direct or indirect and that this specification is not intended to be limiting in this respect. Any reference to attached, fixed, connected or the like may include permanent, removable, temporary, partial, full and/or any other possible attachment option.

No element, component, or method step in the present disclosure is intended to be dedicated to the public regardless of whether the element, component, or method step is explicitly recited in the claims. No claim element herein is to be construed under the provisions of 35 U.S.C. 112(f) unless the element is expressly recited using the phrase “means for.” As used herein, the terms “comprises”, “comprising”, or any other variation thereof, are intended to cover a non-exclusive inclusion, such that a process, method, article, or apparatus that comprises a list of elements does not include only those elements but may include other elements not expressly listed or inherent to such process, method, article, or apparatus.

While various inventive aspects, concepts and features of the disclosures may be described and illustrated herein as embodied in combination in the exemplary embodiments, these various aspects, concepts, and features may be used in many alternative embodiments, either individually or in various combinations and sub-combinations thereof. Unless expressly excluded herein all such combinations and sub-combinations are intended to be within the scope of the present application. Still further, while various alternative embodiments as to the various aspects, concepts, and features of the disclosures—such as alternative structures, configurations, methods, devices, and components, and so on—may be described herein, such descriptions are not intended to be a complete or exhaustive list of available alternative embodiments, whether presently known or later developed. Those skilled in the art may readily adopt one or more of the inventive aspects, concepts, or features into additional embodiments and uses within the scope of the present application even if such embodiments are not expressly disclosed herein. For example, in the exemplary embodiments described above within the Detailed Description portion of the present specification, elements may be described as individual units and shown as independent of one another to facilitate the description. In alternative embodiments, such elements may be configured as combined elements. It is further noted that various method or process steps for embodiments of the present disclosure are described herein. The description may present method and/or process steps as a particular sequence. However, to the extent that the method or process does not rely on the particular order of steps set forth herein, the method or process should not be limited to the particular sequence of steps described. As one of ordinary skill in the art would appreciate, other sequences of steps may be possible.

Additionally, even though some features, concepts, or aspects of the disclosures may be described herein as being a preferred arrangement or method, such description is not intended to suggest that such feature is required or necessary unless expressly so stated. Still further, exemplary or representative values and ranges may be included to assist in understanding the present application, however, such values and ranges are not to be construed in a limiting sense and are intended to be critical values or ranges only if so expressly stated.

The treatment techniques, methods, and steps described or suggested herein or in references incorporated herein may be performed on a living animal or on a non-living simulation, such as on a cadaver, cadaver heart, anthropomorphic ghost, or simulator (e.g., with the body parts, or tissue being simulated).

Any of the various systems, devices, apparatuses, etc. in this disclosure may be sterilized (e.g., with heat, radiation, ethylene oxide, hydrogen peroxide) to ensure they are safe for use with patients, and the methods herein may comprise sterilization of the associated system, device, apparatus, etc.; e.g., with heat, radiation, ethylene oxide, hydrogen peroxide.

1. A method of non-invasively determining continuous total hemoglobin data, comprising:
using a near infra-red spectrophotometric (NIRS) sensing device on a continuous basis to sense a subject's tissue, wherein NIRS signals are produced from the sensing and are representative of total hemoglobin (THb);
providing a body mass index (BMI) value of the subject;
selectively calibrating the NIRS sensing device using a reference absolute THb value acquired from the subject, wherein the selective calibration includes calibrating or not calibrating the NIRS sensing device based on the BMI value; and
determining continuous THb data using the produced NIRS signals.

2. The method of claim 1, wherein the continuous THb data is continuous relative THb data.

3. The method of claim 1, wherein the continuous THb data is continuous absolute THb data.

4. The method of claim 1, further comprising using the BMI value to determine a blood volume of the subject.

5. The method of claim 4, wherein the step of selectively calibrating the NIRS sensing device includes using the BMI value to determine a blood volume of the subject and the subject blood volume is used in the calibrating or not calibrating the NIRS sensing device.

6. The method of claim 1, further comprising determining if a cardiopulmonary bypass (CPB) on the subject has occurred.

7. The method of claim 6, wherein the step of determining if said CPB on the subject has occurred utilizes a signal from a blood pressure measurement device.

8. The method of claim 7, wherein the signal from the blood pressure measurement device is a pulsatile waveform.

9. The method of claim 6, wherein if the occurrence of a CPB is determined, the step of selectively calibrating the NIRS sensing device includes using the BMI value to determine a blood volume of the subject and the subject blood volume is used in the calibrating or not calibrating the NIRS sensing device.

10. A method of non-invasively determining continuous total hemoglobin data during a medical procedure that includes cardiopulmonary bypass (CPB) performed using a CPB device, the method comprising:
using a near infra-red spectrophotometric (NIRS) sensing device on a continuous basis to sense a subject's tissue, wherein NIRS signals are produced from the sensing and are representative of total hemoglobin (THb);
providing a body mass index (BMI) value of the subject;
providing a priming volume value for the CPB device;
determining an occurrence of the CPB;
if the step of determining the occurrence of the CPB determines CPB has occurred, selectively calibrating the NIRS sensing device using a reference absolute THb value acquired from the subject, wherein the selectively calibrating includes a determination to calibrate or not calibrate the NIRS sensing device and the determination to calibrate or not calibrate uses the BMI value and the priming volume value; and
determining continuous THb data using the produced NIRS signals.

11. The method of claim 10, wherein the continuous THb data is continuous relative THb (ΔTHb) data.

12. The method of claim 10, wherein the continuous THb data is continuous absolute THb data.

13. The method of claim 10, further comprising using the BMI value to determine a blood volume of the subject value, and the blood volume is used in the determination to calibrate or not calibrate.

14. The method of claim 10, wherein the step of determining the occurrence of the CPB utilizes a signal from a blood pressure measurement device.

15. A system for determining continuous total hemoglobin data from a subject, comprising:
a near infra-red spectroscopy (NIRS) sensing device configured to sense a tissue region of the subject, and to produce NIRS signals from the sensing;
a controller in communication with the NIRS sensing device, the controller including at least one processor and a memory device configured to store instructions, which instructions when executed cause the controller to:
control the NIRS sensing device to sense a subject's tissue on a continuous basis, and produce NIRS signals from the sensing that are representative of total hemoglobin (THb);
determine a blood volume value for the subject based on an input body mass index (BMI) value for the subject;
selectively calibrating the NIRS sensing device using a reference absolute THb value acquired from the subject, wherein the selective calibration includes calibrating or not calibrating the NIRS sensing device based on the blood volume value; and
determine continuous THb data using the produced NIRS signals.

16. The system of claim 15, wherein the continuous THb data is continuous relative total hemoglobin (ΔTHb).

17. The system of claim 15, wherein the continuous THb data is continuous absolute THb.

18. The system of claim 15, wherein the instructions when executed cause the controller to determine if a cardiopulmonary bypass (CPB) on the subject has occurred.

19. The system of claim 18, wherein the controller is configured to communicate with a blood pressure measurement device and the determination of whether a CPB on the subject has occurred is based on signals from the blood pressure measurement device.

20. A non-transitory computer readable medium comprising software code sections which are adapted to perform a method for non-invasively determining continuous total hemoglobin data, including the steps of:
controlling a near infra-red spectrophotometric (NIRS) sensing device on a continuous basis to sense a subject's tissue, the sensing producing NIRS signals that are representative of total hemoglobin (THb);
determining a blood volume value of the subject using a body mass index (BMI) value;
determining if the subject is undergoing cardiopulmonary bypass (CPB);
if the subject is undergoing CPB, selectively calibrating the NIRS sensing device using a reference absolute THb value acquired from the subject, wherein the selectively calibrating includes a determination to calibrate or not calibrate the NIRS sensing device and the determination to calibrate or not calibrate uses the determined blood volume value; and
determining the continuous THb data using the produced NIRS signals.

21. The non-transitory computer readable medium of claim 20, wherein the continuous THb data is continuous relative THb data.

22. The non-transitory computer readable medium of claim 20, wherein the continuous THb data is continuous absolute THb data.

23. A method of monitoring a patient blood transfusion, comprising:
providing a body mass index (BMI) value of the patient;
using the BMI value to determine the patient's circulatory blood volume;
using a near infra-red spectrophotometric (NIRS) sensing device on a continuous basis to sense the patient's tissue during the transfusion, wherein NIRS signals are produced from the sensing and are representative of a hemoglobin value of the patient's circulatory blood; and
using the BMI value and the NIRS signals in the monitoring of the transfusion.

24. The method of claim 23, wherein the hemoglobin value is continuous total hemoglobin (THb) data.

25. The method of claim 24, wherein the continuous THb data is continuous relative THb (ΔTHb) data.

26. The method of claim 24, wherein the continuous THb data is continuous absolute THb data.

27. A patient blood transfusion monitoring system, comprising:
a near infra-red spectroscopy (NIRS) sensing device configured to sense a tissue region of the patient, and to produce NIRS signals from the sensing; and
a controller in communication with the NIRS sensing device, the controller including at least one processor and a memory device configured to store instructions, which instructions when executed cause the controller to:
control the NIRS sensing device to sense a patient's tissue on a continuous basis and produce NIRS signals from the sensing that are representative of a hemoglobin value of the patient's circulatory blood;
determine a circulatory blood volume value for the patient using a provided body mass index (BMI) value of the patient;
provide information representative of the transfusion on a continuous basis using the NIRS signals and the determined circulatory blood volume.

28. The system of claim 27, wherein the hemoglobin value is continuous total hemoglobin (THb) data.

29. The system of claim 28, wherein the continuous THb data is continuous relative THb (ΔTHb) data.

30. The system of claim 28, wherein the continuous THb data is continuous absolute THb data.

31. A non-transitory computer readable medium comprising software code sections which are adapted to perform a method for monitoring a patient blood transfusion, including the steps of:
determining a patient's circulatory blood volume using a provided body mass index (BMI) value of the patient;
controlling a near infra-red spectrophotometric (NIRS) sensing device on a continuous basis to sense the patient's tissue during the transfusion, wherein NIRS signals are produced from the sensing and are representative of a hemoglobin value of the patient's circulatory blood; and
providing information representative of the transfusion on a continuous basis using the NIRS signals and the determined circulatory blood volume.
